(19) United States
(12) Patent Application Publication
(10) Pub. No.: US 2011/0184289 A1
(43) Pub. Date: Jul. 28, 2011

(54) ULTRASONIC DIAGNOSTIC APPARATUS

(75) Inventors: Mitsuhiro Oshiki, Tokyo (JP); Shinichiro Kishi, Tokyo (JP); Atsushi Suzuki, Tokyo (JP)

(73) Assignee: HITACHI MEDICAL CORPORATION, Tokyo (JP)

(21) Appl. No.: 12/996,095

(22) PCT Filed: Jun. 3, 2009
(86) PCT No.: PCT/JP2009/060112

§ 371 Date: Apr. 13, 2011

(30) Foreign Application Priority Data
Jun. 5, 2008 (JP) 2008-148311

(51) Int. Cl. A61B 8/14

(52) U.S. Cl. 600/443

(57) ABSTRACT

An ultrasonic diagnostic apparatus in accordance with the invention includes: an ultrasonic probe in which multiple ultrasonic vibrators for transmitting/receiving an ultrasonic wave are arranged; a transmitter configured to provide an electric signal to each of the vibrators in the ultrasonic probe, the transmitter providing a square wave signal having any multiple frequency components to the each of the vibrators, causing the vibrators to form an ultrasonic beam; a receiver configured to receive a reception signal obtained by transmitting the ultrasonic beam; and a signal processor configured to form an ultrasonic image based on the reception signal.

### TECHNICAL FIELD

The present invention relates to an ultrasonic diagnostic apparatus that can transmit a square wave and, more particularly, to an ultrasonic diagnostic apparatus including a square wave transmission circuit that can output a transmission signal having multiple frequency components in one transmission.

### BACKGROUND ART

An ultrasonic diagnostic apparatus transmits an ultrasonic wave generated by an ultrasonic vibrator built in an ultrasonic probe to an object to be tested and receives by the ultrasonic vibrator a reflected signal generated by difference in acoustic impedance due to hardness of a tissue of the object to display on a monitor.

Conventionally, an arbitrary waveform amplifier is commonly used to drive the above-described vibrator. On the other hand, as an example of technique not using the arbitrary waveform amplifier, Patent Document 1 discloses a transmission circuit for diagnostic apparatus having a square wave signal amplifier circuit that can suppress the degradation of an image obtained from harmonics generated from within a living body or from contrast agent or the like by reducing harmonics generation.

### Prior Art Document

### Patent Document

Patent Document 1: JP-A-2002-315748

### SUMMARY OF THE INVENTION

### Problems to be Solved by the Invention

However, according to the disclosure of Patent Document 1, the square wave signal output circuit only decreases the duty ratio of each pulse as the distance from the center to the both edges of the amplitude of an input signal increases to suppress the generation of high-frequency components of the envelope shape of the pulse. So, arbitrary waveform generation by square wave signal circuit has not been achieved yet.

It is an object of the invention to provide an ultrasonic diagnostic apparatus that can generate an arbitrary waveform using a square wave signal circuit.

### Means for Solving the Problems

In order to achieve the above object, an ultrasonic diagnostic apparatus in accordance with the invention is characterized by including: an ultrasonic probe in which multiple ultrasonic vibrators for transmitting/receiving an ultrasonic wave are arranged; a transmitter for providing an electric signal to each of the vibrators in the ultrasonic probe, the transmitter providing a square wave signal having any multiple frequency components to the each of the vibrators, causing the vibrators to form an ultrasonic beam; a receiver for receiving a reception signal obtained by transmitting the ultrasonic beam; and a signal processor for forming an ultrasonic image based on the reception signal.

According to the above configuration, an ultrasonic wave having an arbitrary waveform can be generated using a square wave signal circuit in which: the transmitter provides an electric signal to each of the vibrators in the ultrasonic probe, the transmitter providing a square wave signal having any multiple frequency components to the each of the vibrators, causing the vibrators to form an ultrasonic beam; the receiver receives a reception signal obtained by transmitting the ultrasonic beam; and the signal processor forms an ultrasonic image based on the reception signal.

### Advantage of the Invention

According to the invention, it is possible to provide an ultrasonic diagnostic apparatus that can generate an ultrasonic wave having an arbitrary waveform using a square wave signal circuit.

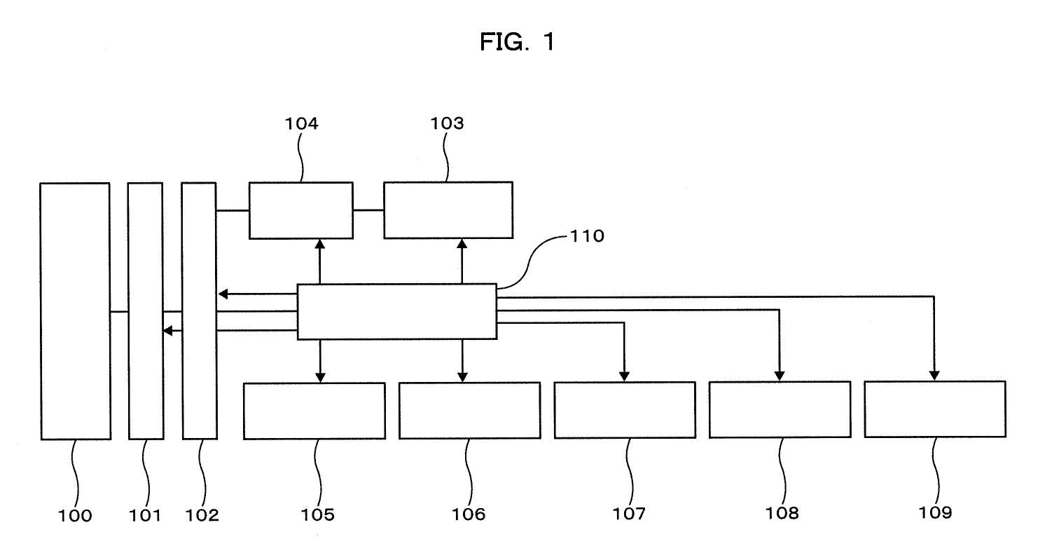

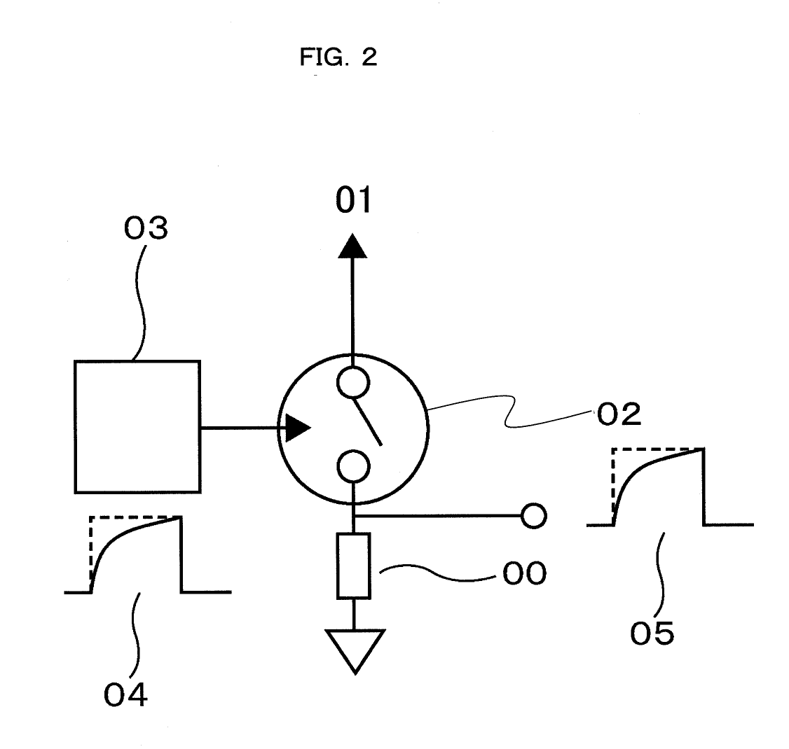

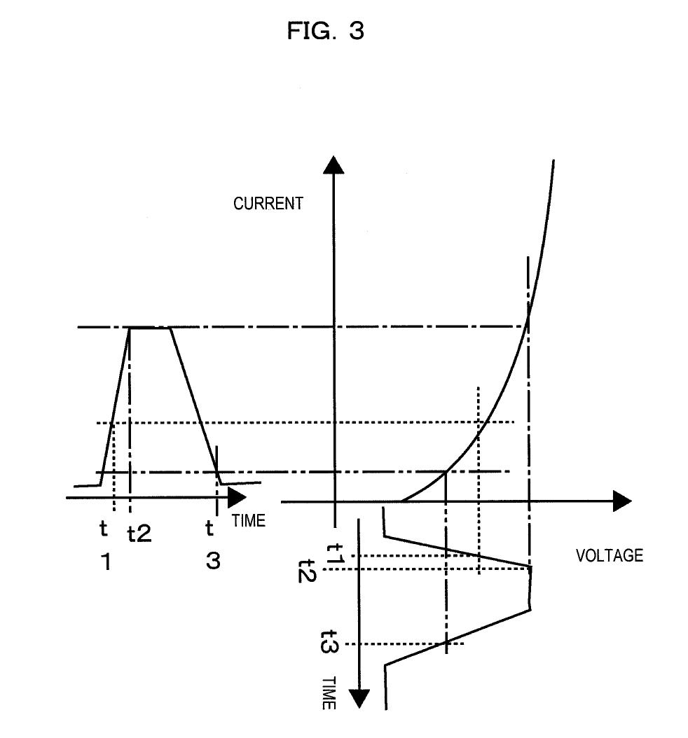

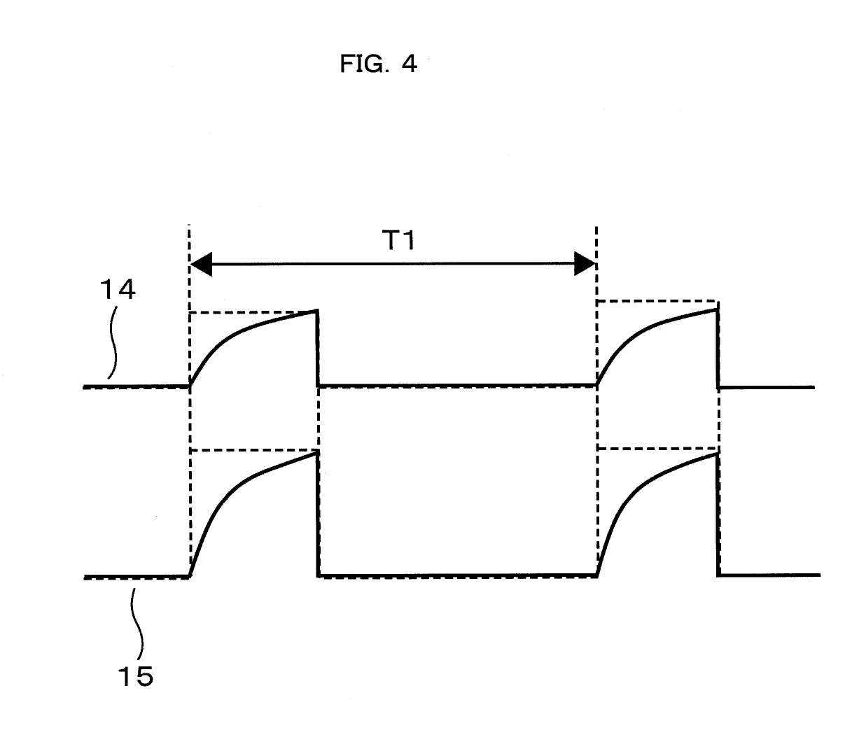

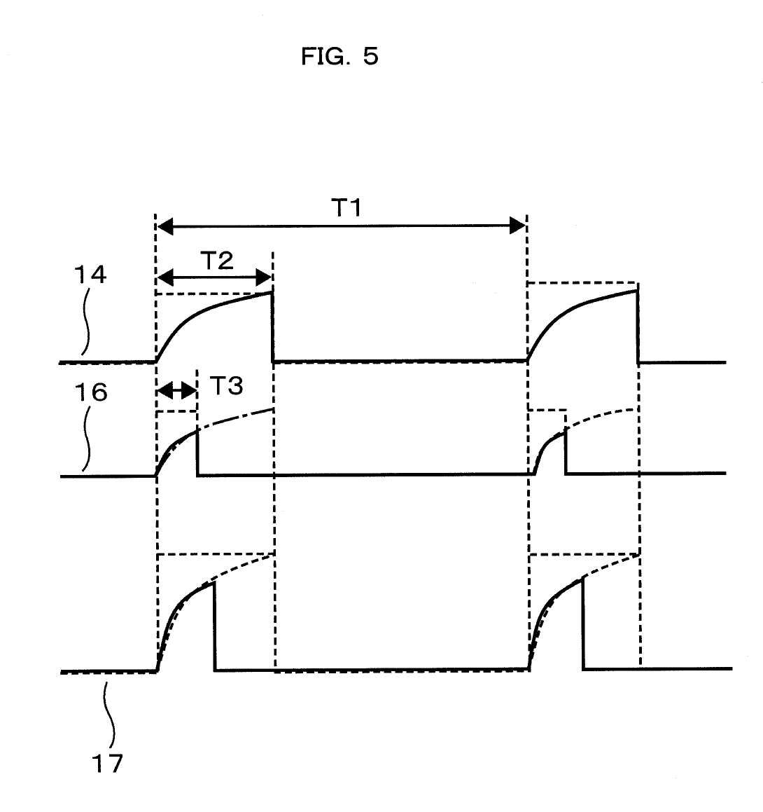

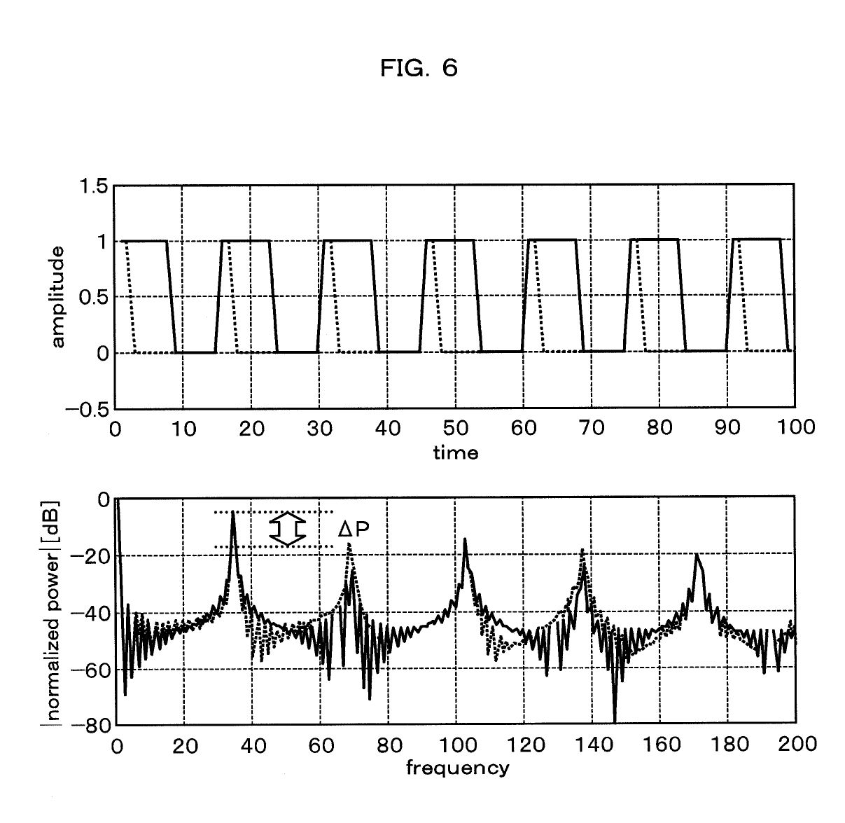

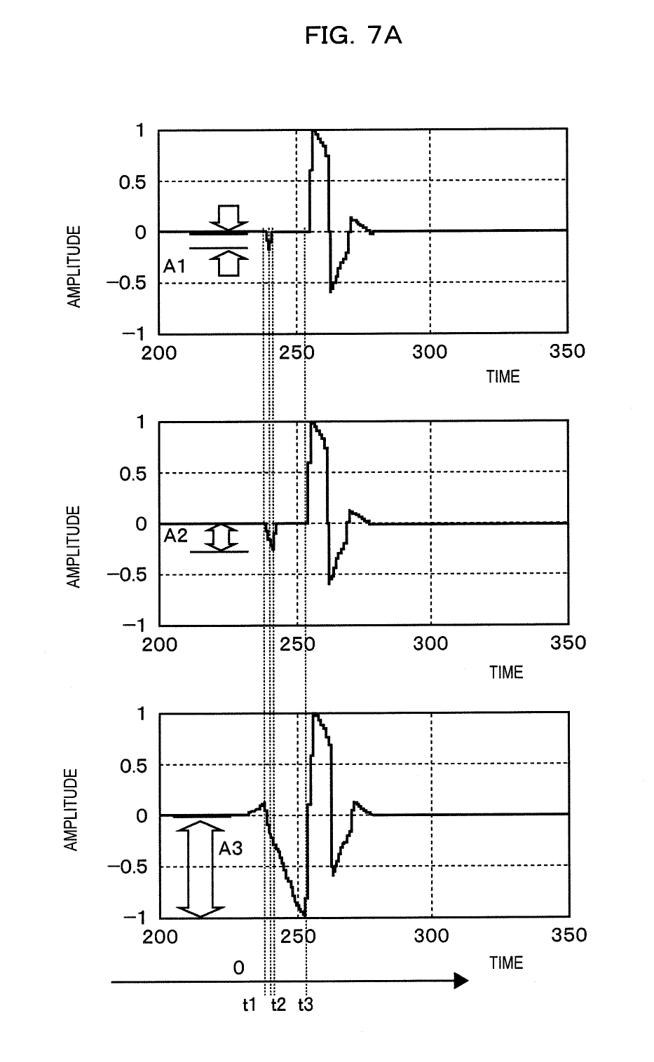

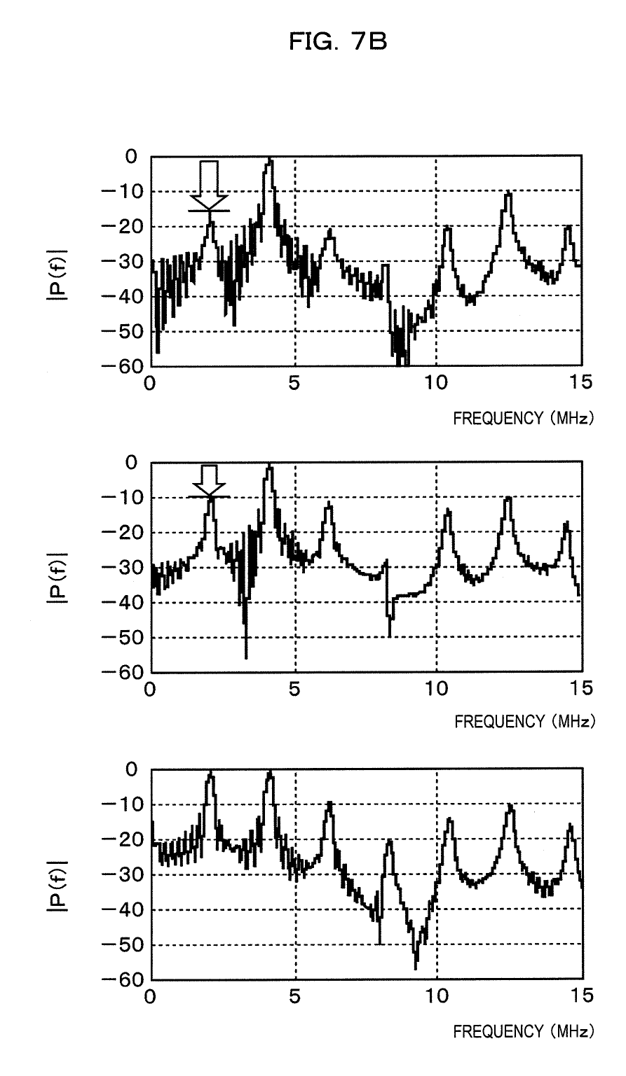

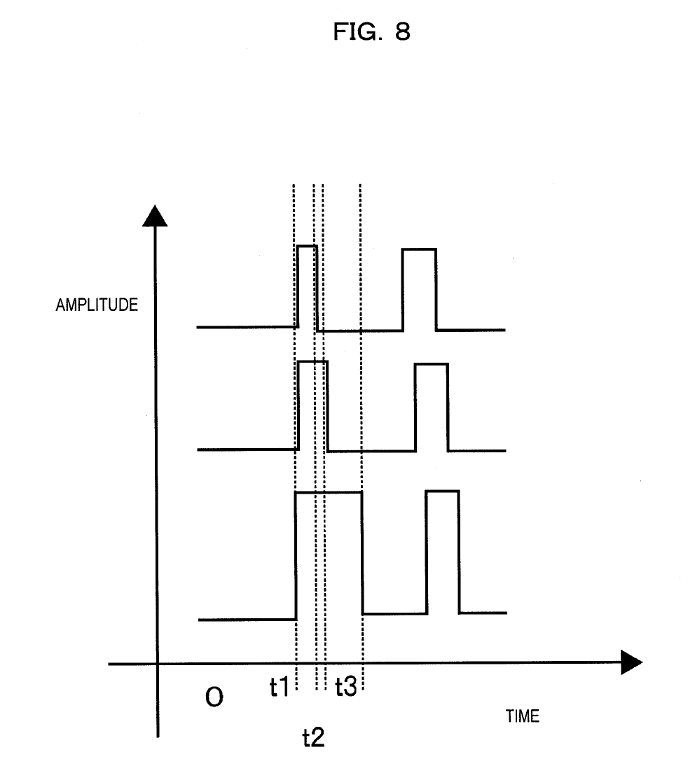

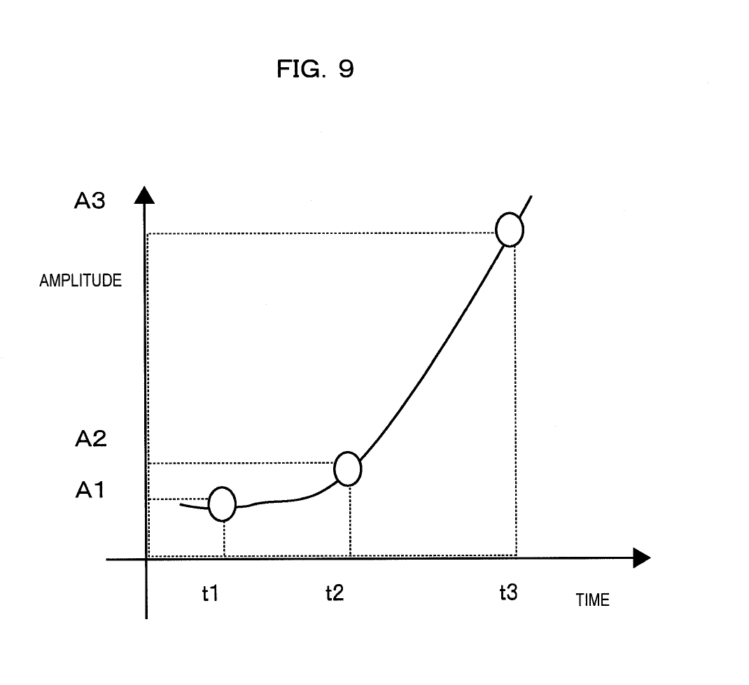

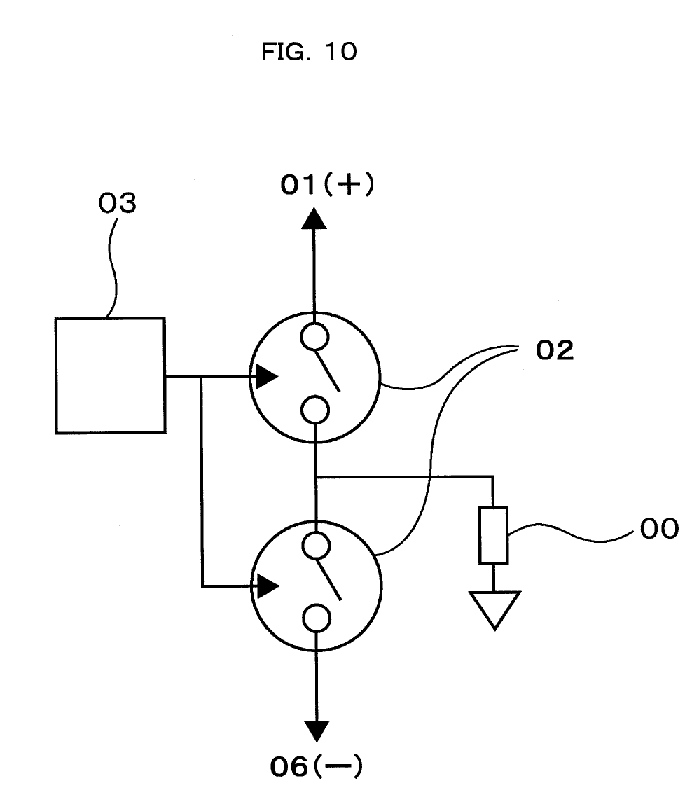

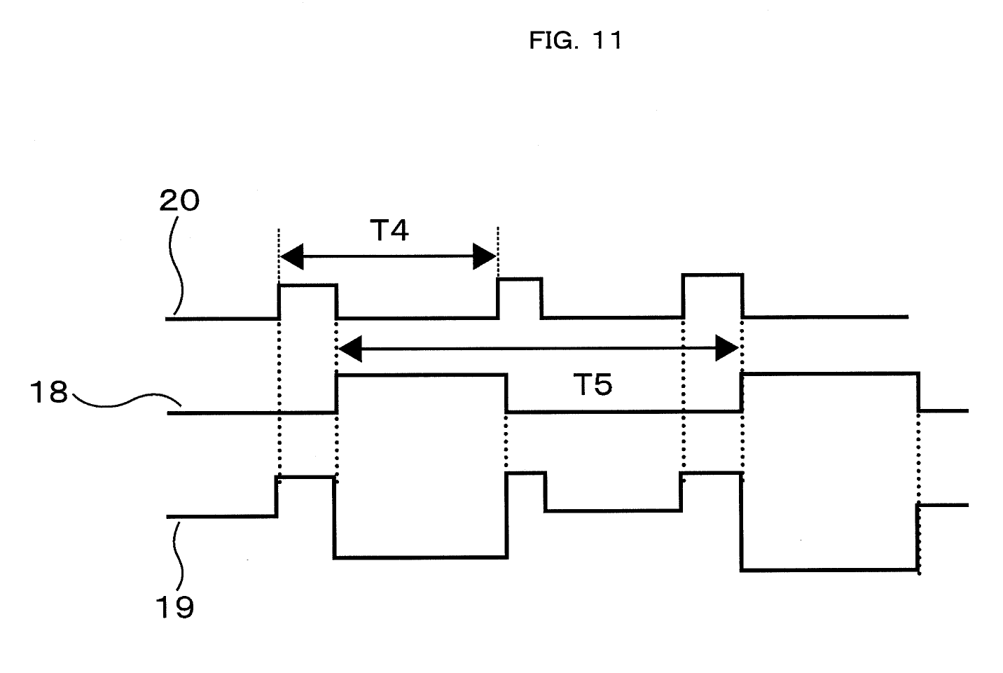

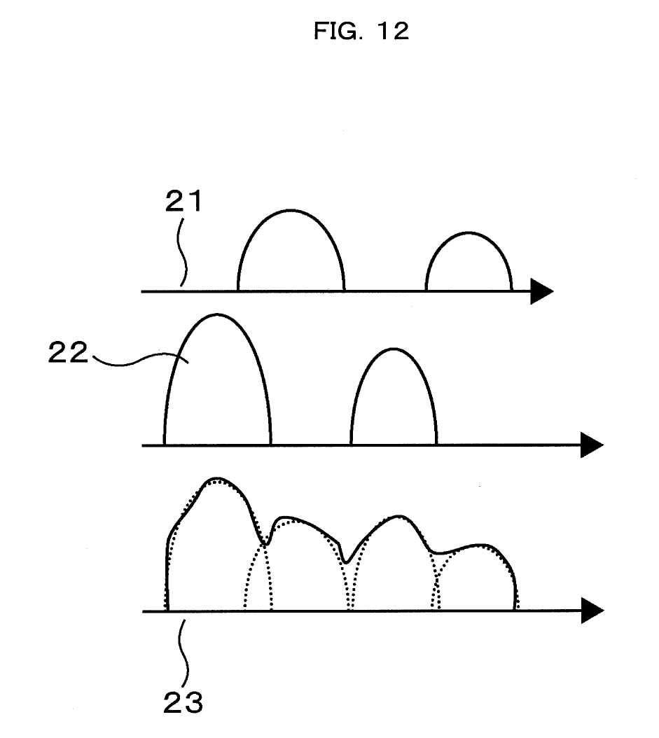

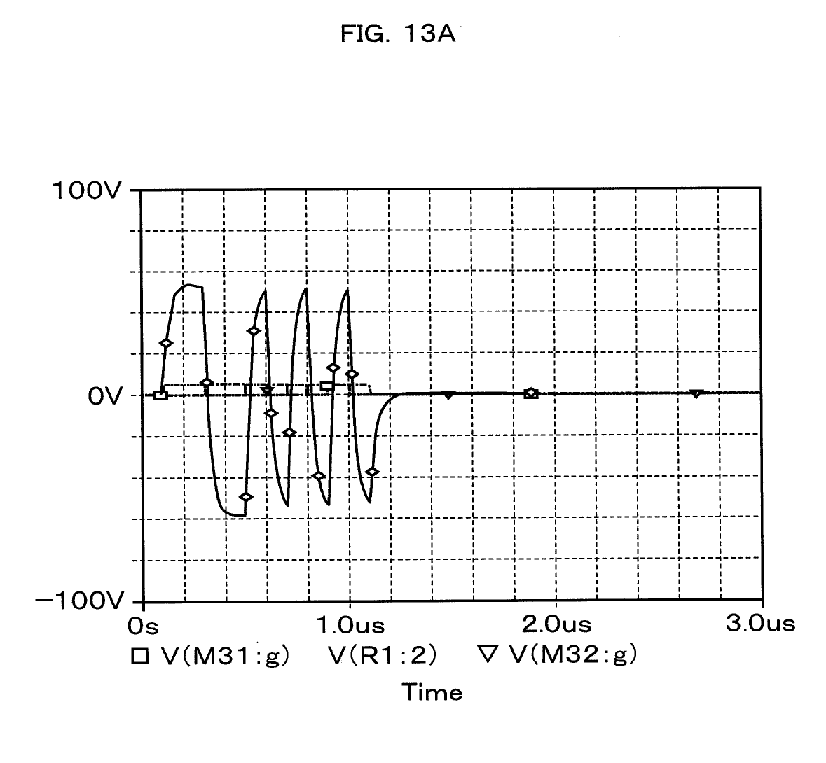

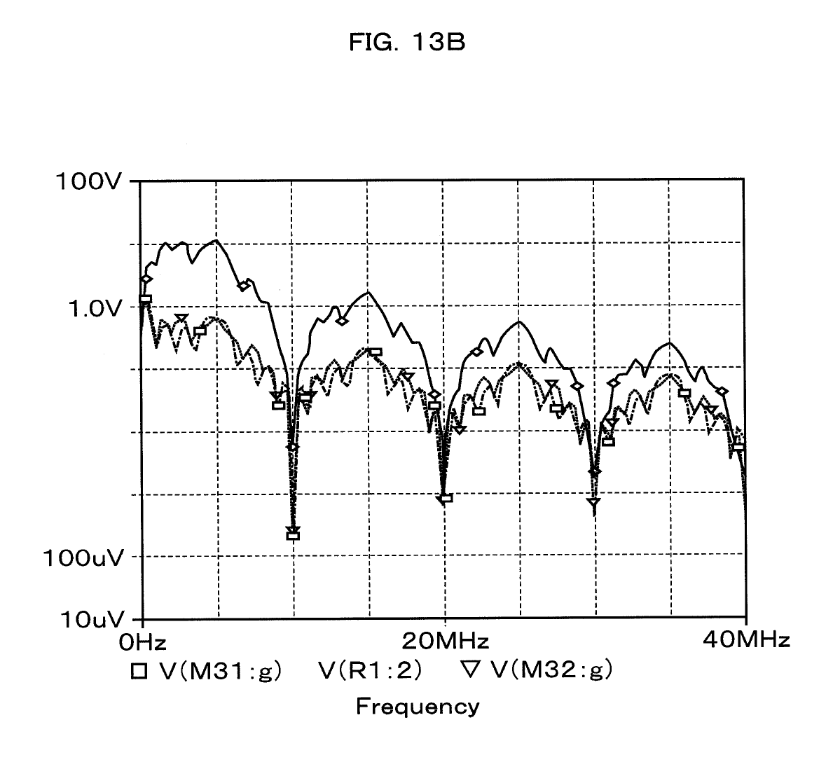

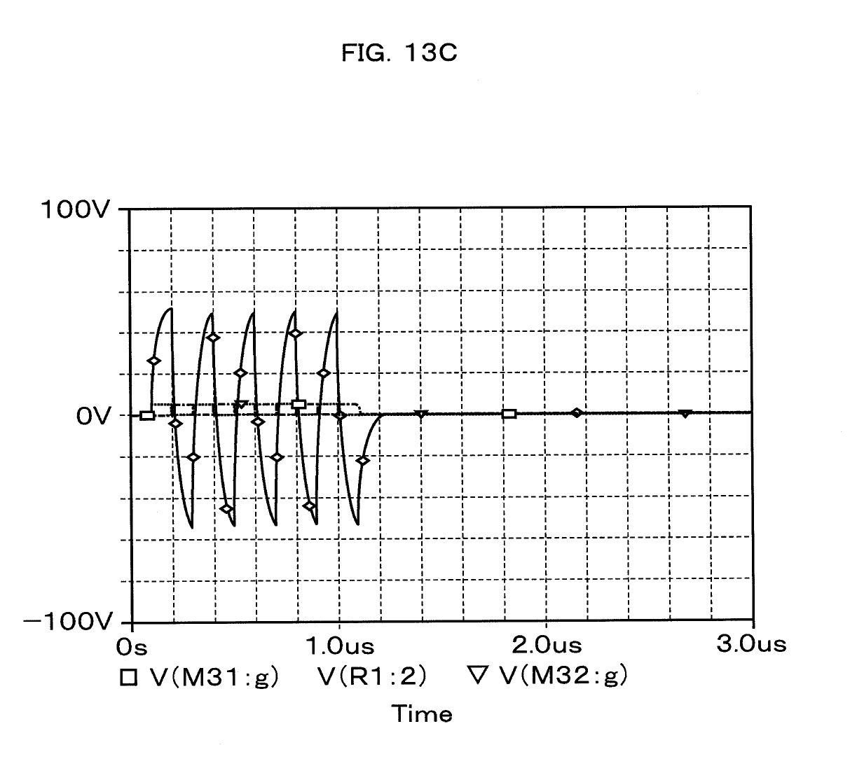

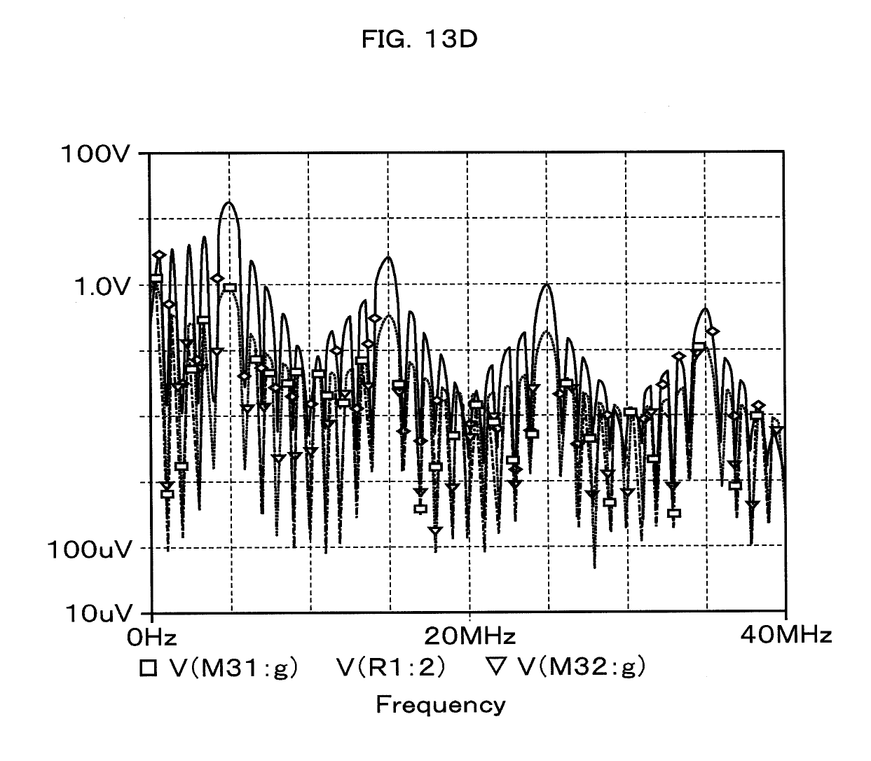

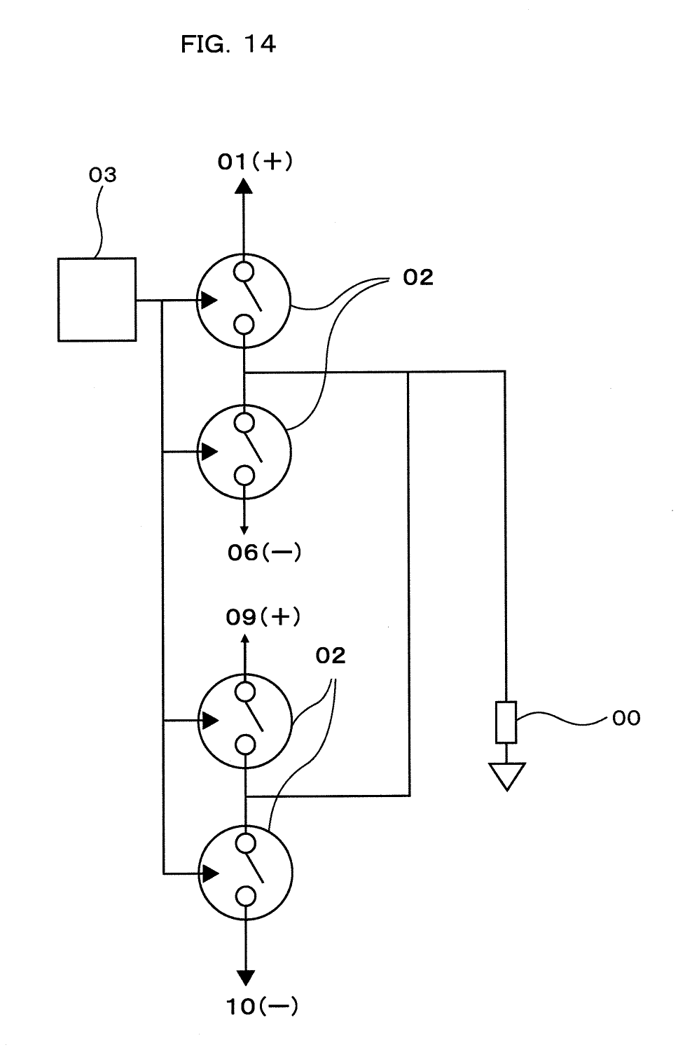

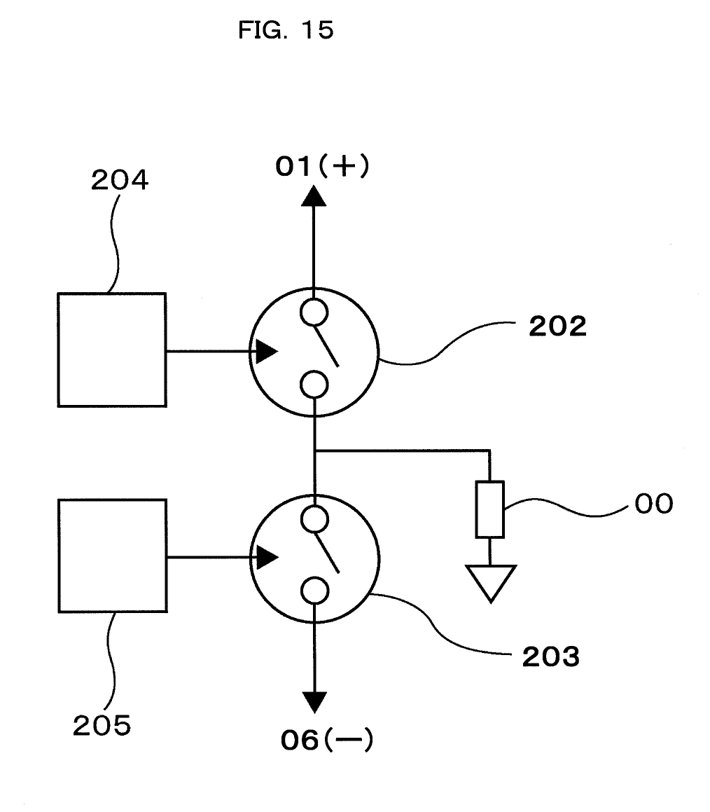

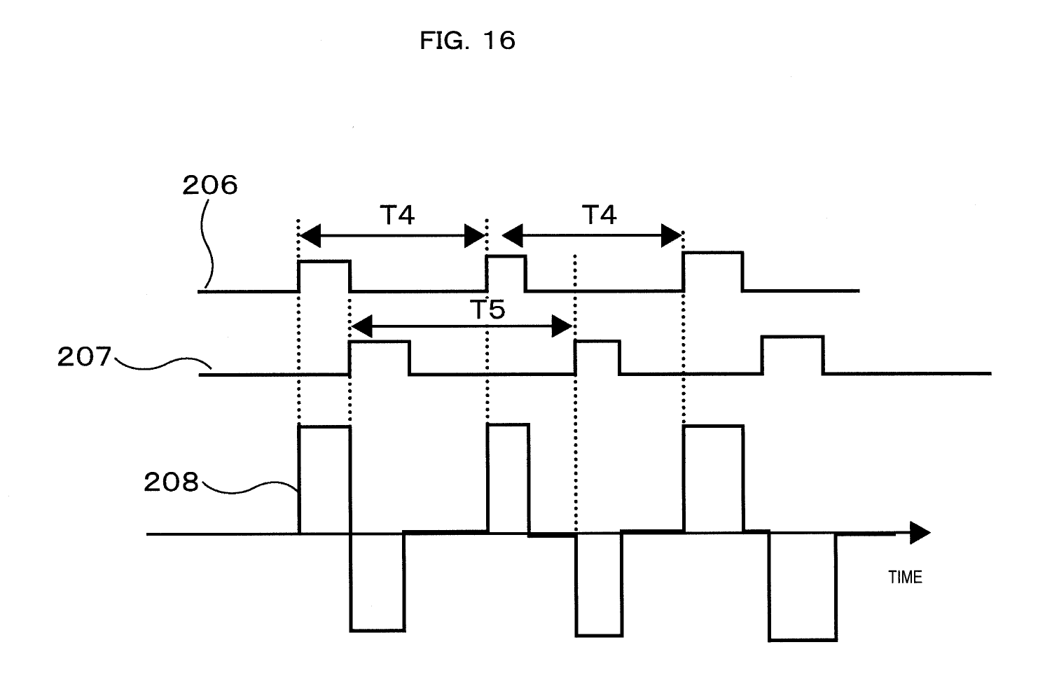

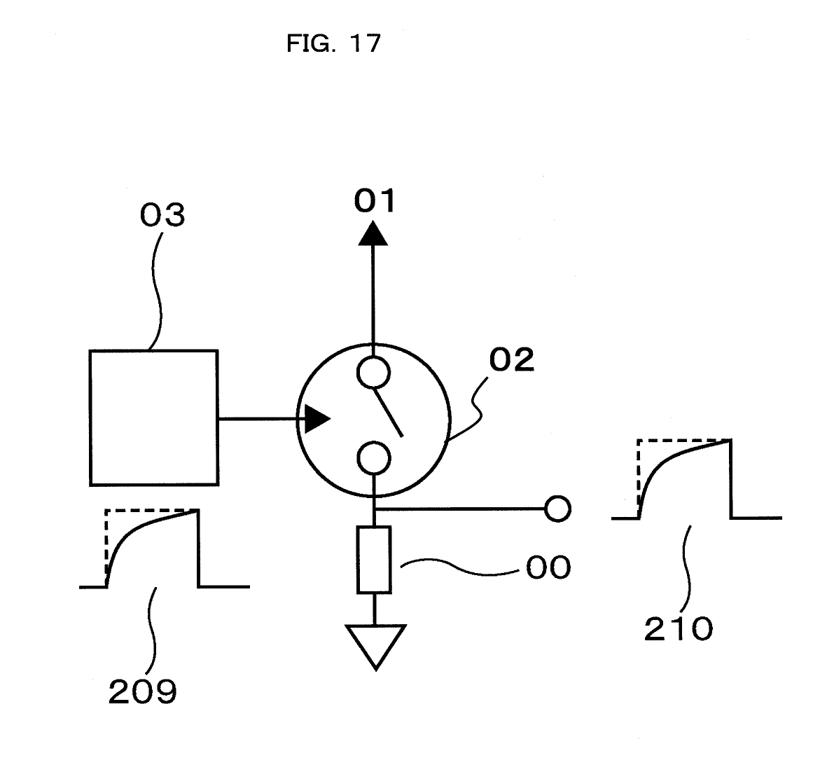

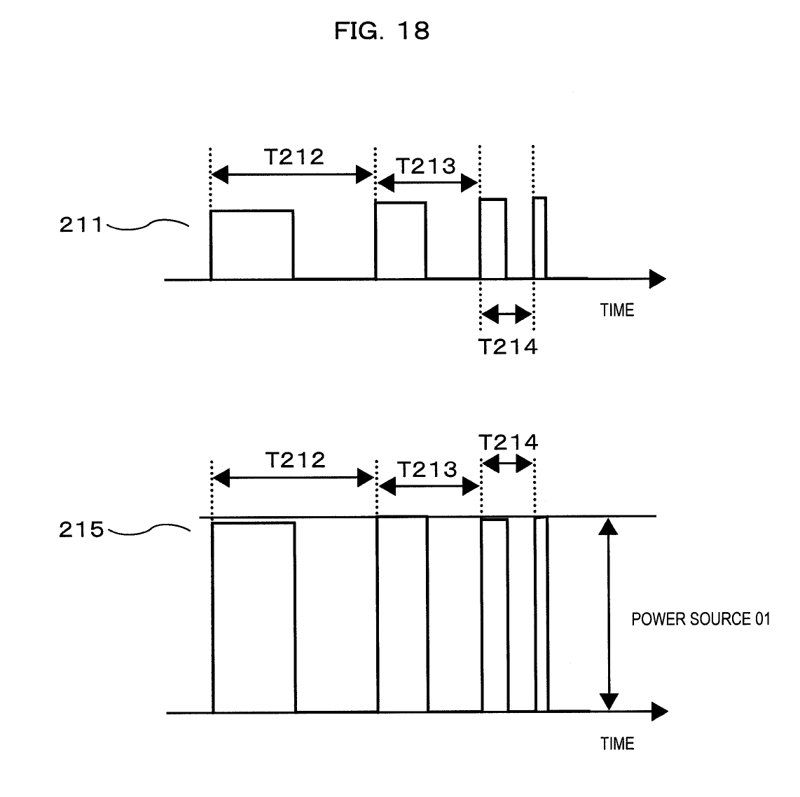

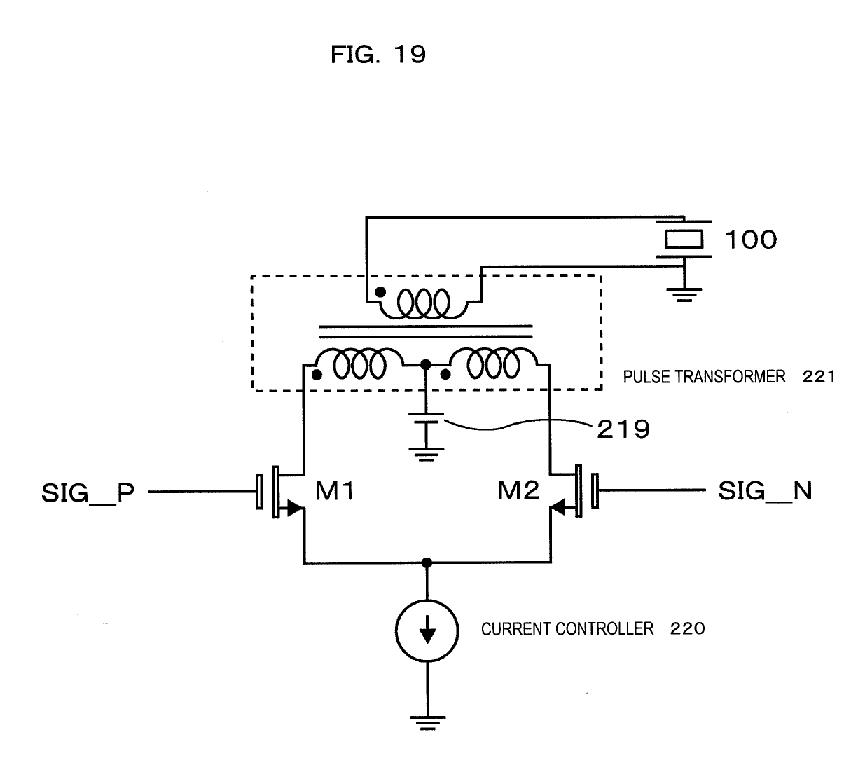

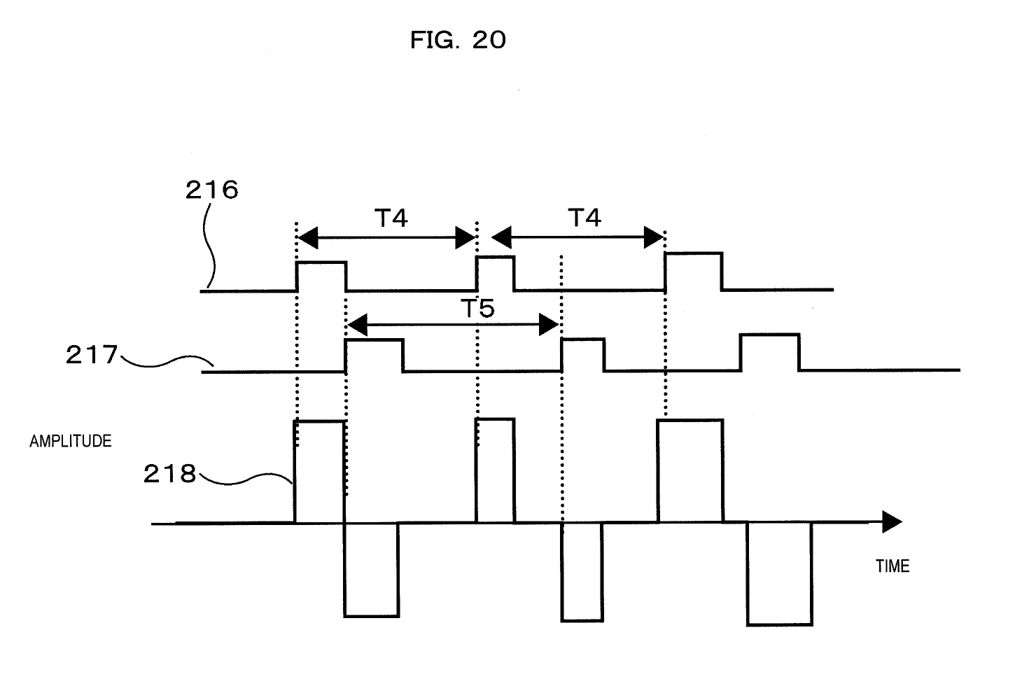

### BRIEF DESCRIPTION OF THE DRAWINGS

[FIG. 1] A schematic block configuration diagram of an ultrasonic diagnostic apparatus in accordance with the invention.

[FIG. 2] A configuration diagram of a square wave transmission circuit in accordance with a first embodiment.

[FIG. 3] A current-voltage diagram of a switching element (FET) shown in FIG. 2.

[FIG. 4] An illustration showing the control timing of the square wave transmission circuit shown in FIG. 2.

[FIG. 5] An illustration showing the control timing of the square wave transmission circuit in accordance with the first embodiment.

[FIG. 6] An illustration showing the correlation between the input signal duty ratio and the output amplitude level in the square wave transmission circuit in accordance with the first embodiment.

[FIG. 7A] An illustration showing the correlation between the input signal duty ratio and the output amplitude level in the square wave transmission circuit in accordance with the first embodiment.

[FIG. 7B] An illustration showing the correlation between the input signal duty ratio and the output amplitude level in the square wave transmission circuit in accordance with the first embodiment.

[FIG. 8] An illustration showing the correlation between the input signal duty ratio and the output amplitude level in the square wave transmission circuit in accordance with the first embodiment.

[FIG. 9] An illustration showing the correlation between the input signal duty ratio and the output amplitude level in the square wave transmission circuit in accordance with the first embodiment.

[FIG. 10] A configuration diagram of a square wave transmission circuit in accordance with a second embodiment.

[FIG. 11] An illustration showing the control timing of the square wave transmission circuit in accordance with the second embodiment.

[FIG. 12] An illustration showing the frequency distribution of the output signal of the square wave transmission circuit in accordance with the second embodiment.

[FIG. 13A] Graphs showing a specific example of the input and output signals and frequency responses thereof in the square wave transmission circuit in accordance with the second embodiment.

[FIG. 13B] Graphs showing a specific example of the input and output signals and frequency responses thereof in the square wave transmission circuit in accordance with the second embodiment.

[FIG. 13C] Graphs showing a specific example of the input and output signals and frequency responses thereof in the square wave transmission circuit in accordance with the second embodiment.

[FIG. 13D] Graphs showing a specific example of the input and output signals and frequency responses thereof in the square wave transmission circuit in accordance with the second embodiment.

[FIG. 14] A configuration diagram of a square wave transmission circuit in accordance with a third embodiment.

[FIG. 15] A configuration diagram of a square wave transmission circuit in accordance with a fourth embodiment.

[FIG. 16] An illustration showing the input and output waveforms of a square wave transmission circuit in accordance with the fourth embodiment.

[FIG. 17] A configuration diagram of a square wave transmission circuit in accordance with a fifth embodiment.

[FIG. 18] An illustration showing the input and output waveforms of a square wave transmission circuit in accordance with the fifth embodiment.

[FIG. 19] A configuration diagram of a square wave transmission circuit in accordance with a sixth embodiment.

[FIG. 20] An illustration showing the input and output waveforms of a square wave transmission circuit in accordance with the sixth embodiment.

### MODE FOR CARRYING OUT THE INVENTION

Specific embodiments of the invention are described below with reference to the drawings. Note that, in the description, a means may be referred to as “circuit” or “section.” For example, a control means may be referred to as “control circuit” or “control section.”

FIG. 1 is a block diagram showing an entire configuration of an ultrasonic diagnostic apparatus for describing the specific embodiments.

The ultrasonic diagnostic apparatus includes: an ultrasonic probe 100 having multiple vibrators; an element selector 101 configured to select an element of the multiple vibrators; a transmission/reception separator 102; a transmission processor 103 configured to form and transmit a transmission signal; a transmitter 104; a reception amplifier 105 configured to amplify a received signal from the ultrasonic probe 100; a phasing addition processor 106; a signal processor 107 configured to perform signal processing such as logarithmic processing on a signal from the phasing addition processor 106; a scan converter 108 configured to scan-convert from ultrasonic scanning to display scanning using a signal from the signal processor 107; a display monitor 109, including a CRT, liquid crystal display or the like, configured to display an image data from the scan converter 108; and a controller 110 configured to control these components.

The transmission/reception separator 102 switches the signal direction depending on whether transmission or reception is occurring. The transmitter 104 provides a drive signal to the multiple vibrators (not shown) in the ultrasonic probe 100 in order to transmit an ultrasonic wave to within an object to be tested. The transmission processor 103 includes a known pulse generator circuit, a known amplifier circuit and a known transmission delay circuit to provide a transmission signal to the transmitter 104.

The multiple vibrators convert reflected waves (echoes) reflected from within the object due to an ultrasonic wave transmitted into the object to electric signals (received signals). The phasing addition processor 106 uses the received signals to form and output an ultrasonic beam signal as if having received from a predetermined direction. The phasing addition processor 106 includes a known reception delay circuit and a known adder circuit.

The signal processor 107 performs logarithmic conversion, filtering, gamma (γ) correction and the like as preprocessing for imaging a received signal output from the phasing addition processor 106.

The scan converter 108 accumulates a signal output from the signal processor 107 for each ultrasonic beam scanning to form an image data and outputs the image data according to the scanning of an image display device, that is, performs scan conversion from ultrasonic scanning to display scanning.

The display monitor 109 is a display device for displaying as an image an image data (converted to a luminance signal) output from the scan converter 108.

The controller 110 is a central processing unit (CPU) for directly or indirectly controlling the above-described components to perform ultrasonic transmission/reception and image displaying.

In the configuration of this ultrasonic diagnostic apparatus, the ultrasonic probe 100 is touched to an area to be tested of the object (not shown), then, a scan parameter such as transmission focus depth is input to the controller 110, and then, an instruction to start ultrasonic scanning is input. The controller 110 controls the components to start ultrasonic scanning.

The controller 110 outputs to the element selector 101 and the transmission processor 103 an instruction to select a vibrator to be used in the first transmission, an instruction to output a drive pulse and an instruction to set a delay time according to the transmission focus depth. When these instructions are executed, the transmission processor 103 provides a drive pulse to the transmitter 104 via a transmission delay circuit (not shown). The transmitter 104 amplifies the drive pulse to a sufficient amplitude for driving the multiple vibrators in the probe 100 and provides the amplified drive pulse to the ultrasonic probe 100.

Of the vibrators in the ultrasonic probe 100, vibrators selected by the element selector 101 and the transmitter 104 that provides a transmission signal are connected via the transmission/reception separator 102. When the drive pulse is input, the vibrators vibrate at predetermined frequencies and sequentially transmit an ultrasonic wave into the object.

When the ultrasonic wave is transmitted into the object, a portion of the wave is reflected by a surface of a tissue or organ in a living body at which acoustic impedance changes, toward the ultrasonic probe 100 as echoes. The controller 110 controls the reception chain to receive the echoes.

Specifically, first, upon finishing the transmission, the element selector 101 performs switching selection to connect a vibrator for reception with the phasing addition processor 106. With this vibrator switching selection, control of reception delay time is performed on the phasing addition processor 106.

The received signals delayed by reception delay circuits are phased and added by the phasing addition processor 106 into a reception beam signal that is output to the signal processor 107. The signal processor 107 performs the above-described processing on the received signal input from the phasing addition processor 106 and outputs the processed signal to the scan converter 108. The scan converter 108 stores the input signal in a memory (not shown) and reads to output the stored contents to the display monitor 109 according to a synchronization signal for displaying. Upon finishing the above operation, the controller 110 changes the direction of ultrasonic transmission/reception to perform the second round of the operation, and then performs the third round and so on. In this way, the controller 110 sequentially changes the direction of ultrasonic transmission/reception to repeat the above operation.

In the above described configuration, the invention relates generally to the transmission circuit chain and, in particular, to the transmission processor 103, the transmitter 104 and the controller 110. Now, embodiments relating to the transmission circuit chain are described below with reference to the drawings.

### First Embodiment

FIG. 2 shows a configuration of a square wave transmission circuit having a single power source used in a first embodiment.

As shown in FIG. 2, the square wave transmission circuit includes: a power source 01 configured according to a voltage applied to a vibrator 00 arranged in the ultrasonic probe 100; a switch element 02 such as a field effect transistor (FET); and a control unit 03 for ON/OFF controlling the switch element 02. In general, in the transmission circuit for ultrasonic diagnostic apparatus, in order to generate an ultrasonic signal sufficient for observing within a living body from an ultrasonic vibrator, a hundred and several tens of volts of electric signal needs to be applied. In order to achieve this, in the transmission circuit, a switch element capable of conducting or interrupting current (turning to ON or OFF) according to a control voltage, such as a high-voltage FET in general, is used.

FIG. 3 shows the output current against the input voltage of a common FET. In the FET, the drain output current has a certain relation with the gate input voltage. FIG. 4 shows an operation timing of the square wave transmission circuit shown in FIG. 1. The broken line shows a theoretical waveform. The solid line shows a realistic waveform.

Suppose, as shown in FIG. 2, an input signal 04 as control signal is applied to the switch 02 by the control unit 03. In order to turn the switch 02 to ON, the input signal 04 as control signal is set to H (high)-state (the same shall apply hereinafter). Thus, in FIG. 4, a control signal 14 showing the switching timing of the switch 02 shows that the switch is turned to ON twice. The control unit 03 is directly or indirectly controlled by the transmission processor 103 and the controller 110.

The input signal 04 (14) is intended to be a square wave as shown by the broken line. However, in reality, it becomes a distorted square wave as shown by the solid line under the influence of the input capacitance of the circuit and the like. Then, the waveform of the output signal 05 (15) as timing signal depends on the input signal as described above. The shape of the output waveform is further influenced by the threshold voltage and output load of an FET element used in the switch circuit. Although the input signal 14 is designed according to the drive capability of a circuit for driving the switch 02, this drive capability is assumed to be constant hereinafter.

The output signal 15 as timing signal shows the waveform of a voltage applied to the vibrator 00. When the control signal 14 is in H-state, the switch 02 is turned to ON, then the power source 01 supplies current to the vibrator 00. Thus, the maximum potential of the vibrator 00 is almost the same as the potential of the power source 01, then a signal for driving an ultrasonic wave is applied. The vibrator 00 performs electroacoustic conversion by this applied voltage to transmit an ultrasonic signal into the living body.

As shown in FIG. 4, the frequency of the square signal shown by the broken line of the control signal 14 is determined by T1 in the figure. When the control signal 14 that is input is in H-state, the timing signal 15 is output. The control signal 14 that is input becomes a distorted square wave as shown by the solid line under the influence of the capacitance in the circuit and the like. The timing signal 15 that is output also has a distorted waveform depending on the capacitance of a load of the vibrator 00 and the like.

In the square wave transmission circuit in accordance with this embodiment, as shown by a signal 16 in FIG. 5, T2, the duration in which the switch 02 is turned to ON of the period T1 of the control signal 14 that is input is changed to T3. In other words, the duty ratio of the waveform is changed from T2/T1 to T3/T1. When an input voltage for causing the switch 02 to supply an output current necessary for fully driving the output load cannot be applied due to the change of the duty ratio, the amplitude of the timing signal 17 that is output is limited, providing an effect equivalent to change of the output amplitude.

In other words, the change of the duty ratio in this embodiment controls the square wave transmission circuit to variably control the duty ratio in the period at which the transmitter provides the square wave signal to the vibrator or to variably change from a first ON-duration set in the switch to a second ON-duration that is different from the first ON-duration in the period at which the transmitter provides the square wave signal to the vibrator.

In this embodiment, as a result, changing the duty ratio of the input signal without multiple power sources can variably change the output amplitude equivalently without changing the signal frequency.

FIG. 6 shows an example of the output waveform amplitude changed by changing the duty ratio using this embodiment. The upper portion of FIG. 6 shows the output signal waveform changed by changing the duty ratio, and the lower portion shows the frequency response of the output signal. According to the example shown in FIG. 6, it has been recognized that reducing the duty ratio to about ¼ can reduce the normalized power by ΔP.

As seen from the above-described embodiment, using a single power source and changing the duty ratio of a positive input signal can change the output waveform amplitude. However, the same also applies to a positive and negative input signal. FIGS. 7A and 7B show how the output amplitude and frequency response vary when the pulse width and duty ratio of the first negative wave of the a transmission waveform of the ultrasonic diagnostic apparatus are changed. In FIGS. 7A and 7B, the input signal is a mixture of two frequencies, and, in this example of three waves of waveform, the first half (1.5 waves) consists of the lower frequency, and the second half (1.5 waves) consists of the higher frequency. In this example, the pulse width of the negative waveform of the input signal is changed from t1 to t3 (thus, the duty ratio is changed) as shown in FIG. 8. Thus, the controller divides the period at which the transmitter provides the square wave signal to the vibrator and provides the vibrator with multiple signals having different frequencies in those respective divided durations of the period to variably control the duty ratio. As shown in FIG. 9, it has been recognized that, when the pulse width is changed from t1 to t3, the output amplitude changes from A1 to A3.

### Second Embodiment

Next, a second embodiment, a case of inputting a positive and negative input signal, is described with reference to FIGS. 10, 11 and 12. This embodiment is a square wave transmission circuit as shown in FIG. 10, in which two power sources—positive power source 01 and a negative power source 06—are provided; a signal having different frequencies between the positive and negative sides of the signal is input; and this signal can be amplified to be output. FIG. 11 is a timing chart of this embodiment. In FIG. 11, a waveform 20 is a waveform of a control signal for one switch circuit 02 connected to the positive power source 01. The signal period of the waveform 20 is set to T4, then the center frequency of the signal is 1/T4. On the other hand, a waveform 18 is a waveform of a control signal for the other switch circuit 02 connected to the negative power source 06. The signal period of the waveform 18 is T5, then the center frequency of the signal is 1/T5. The control signals 18 and 20 are generated by a control unit 03.

As a result of the above, as shown in FIG. 12, an output signal 19 shown in FIG. 11 has a frequency component 21 of 1/T4 in the positive amplitude portion and a frequency component 22 of 1/T5 in the negative amplitude portion. Then, the frequency distribution 23 of the combined output signal 19 is obtained by adding the frequency component 21 and the frequency component 22. This allows even the square signal transmission circuit to output a signal having multiple center frequencies in one transmission and to be used in an ultrasonic diagnostic apparatus that images by tissue harmonic imaging. Furthermore, as seen from FIG. 12, also in this embodiment, the relation between the duty ratio and amplitude of the signal shown by the first embodiment is maintained, so the frequency component of the negative signal 18 with a larger duty ratio is larger.

Note that, in tissue harmonic imaging, the transmission signal may be generated using the technique of the invention and applied to, for example, WO2007/111013.

FIG. 13A shows the output waveform against the input signal having a frequency component varying with time. FIG. 13B shows the frequency distribution of the output waveform. On the other hand, FIG. 13C shows the output waveform of the same circuit against the input signal having a constant frequency. FIG. 13D shows the frequency distribution of the output waveform. It can be recognized that, when the frequency is variably changed with time, the frequency distribution of the output waveform spreads widely.

Thus, variably changing the frequency of the input waveform with time can variably change the output waveform amplitude of the signal the main component of which has the variably changed frequency.

### Third Embodiment

Next, a square wave transmission circuit in accordance with a third embodiment is shown in FIG. 14. This square wave transmission circuit has multiple pairs of positive and negative power sources and the output amplitude is changed. The multiple pairs of power sources enables finer tuning of the waveform than one pair of positive and negative power sources. It will be obvious that, also in this embodiment, a control unit 03 controls switches 02 each connected to the respective power sources 01, 06, 09 and 10 to change the duty ratio of the above-described input signal, enabling the amplitude control.

### Fourth Embodiment

A fourth embodiment is similar to the second embodiment in that a square wave transmission circuit is provided in which a signal having different frequencies between the positive and negative sides of the signal is input and this signal can be amplified to be output. However, this embodiment is different from the second embodiment in that separate control units 204 and 205 are provided in place of the single control unit 03. Now, the fourth embodiment is described below with reference to FIGS. 15 and 16.

As shown in FIG. 15, this embodiment has a circuit configuration including two power sources—a positive power source 01 and a negative power source 06—, corresponding switches 202 and 203, and the control units 204 and 205. The positive signal of the output signal of this circuit is output by the switch 202 connected to the power source 01 having a positive power source value, and the negative signal is similarly output by the switch 203 connected to the power source 06 having a negative power source value. Signals input to the switches 202 and 203 are generated by the transmission processor 103 shown in FIG. 1 and input to the switches 202 and 203 via the control units 204 and 205, respectively.

Of the signals input to the switches, a signal 206 having a period of T4 is input to the switch 202 and a signal 207 having a period of T5 is input to the switch 203. Note that T4≠T5. The signals 206 and 207 input to the switches 202 and 203 have a low amplitude. So, as described with reference to FIG. 1, in order to drive the probe 100 to transmit an ultrasonic wave sufficient for obtaining a signal from the living body, the signals 206 and 207 are amplified to the amplitude of the high-voltage power sources 01 and 06 by the switches 202 and 203, respectively. Accordingly, the signals output from the switches 202 and 203 (thus, the signal output from the transmitter 104) have the same frequencies as those of the switch input signal 206 and 207 and the same amplitudes (maximum amplitudes) as the voltages of the power sources 01 and 06.

Because of T4≠T5, the output signal has a combination of two frequencies rather than a single frequency. An example of the output signal is shown by a signal 208 in FIG. 16. The signal having the period of T4 is output on the positive side, and the signal having the period of T5 is output on the negative side.

### Fifth Embodiment

Next, a transmission circuit for ultrasonic diagnostic apparatus in accordance with a fifth embodiment is described with reference to FIG. 17. In this transmission circuit, the frequency of the input signal can be variably changed in time direction (or with time), and this input signal can be amplified to be output.

A circuit configuration, similarly to that shown in FIG. 2, including a single switch circuit 02 and a single power source 01 is described. For example, when an input signal 209 is input from a control unit 03, an output signal 210 having the same period as that of the signal 209 is output. Depending on the connection with the power source, the phase of the output signal may be inverted.

Suppose, in this transmission circuit configuration, the frequency of the input signal 209 is changed with time, as shown by a waveform 211 in FIG. 18. For example, the change is such that the periods of the first, second and third waves are T212, T213 and T214, respectively. Suppose, for example, T212>T213>T214 (T212≠T213≠T214 would be enough).

Then, as previously described, a signal shown by a waveform 215 appears as the output signal 210 of the transmission circuit, which has the signal amplitude changing to the value of the power source 01 and the frequency changing with time in a way similar to the input signal 209. Thus, the frequency of the output waveform varies with time.

### Sixth Embodiment

The switch circuit has been described above by illustrating the configurations shown in FIGS. 2, 15 and the like. However, the arrangement of power sources and the like are not limited to the above. For example, as shown in FIG. 19, a circuit including a pulse transformer 221 and a single type power source may be used. In this circuit, positive and negative signals are formed by FETs M1 and M2, respectively. The polarity is determined by the polarities (winding directions) of the portions of the pulse transformer 221 connected to M1 and M2 and the polarity (winding direction) of the portion of the pulse transformer 221 connected to the probe 100.

With reference to this circuit, the operation of this embodiment is described by taking an example of an input signal having different frequencies between the positive and negative sides of the signal.

In the circuit of this embodiment, SIG_N and SIG_P in FIG. 19 are provided as a signal input section. The switch section corresponding to the above-described switch 02 is the FETs M1 and M2. The polarities of the portions of the pulse transformer connected to the switches M1 and M2 are opposite with respect to a power source 219 (In the figure, a black circle shows a polarity. The winding of the reactance forming the pulse transformer starts from ). Suppose that waveforms 216 and 217 shown in FIG. 20 are applied as input signals to SIG_P and SIG_N, respectively. When the input signal 216 is in H-state, M1 is turned to ON. When the input signal 217 is in H-state, M2 is turned to ON. Current flows from the power source 219 through the element in ON-state and a current controller 220 to the ground. The current controller 220 controls the amount of current flowing through the switch M1 or M2 when in ON-state.

Suppose that the turn ratio of the pulse transformer 221 shown in FIG. 19 is N1:N2:N3. N1, N2 and N3 are the numbers of turns of the reactances connected to M1, M2 and the vibrator 100, respectively.

Assuming that the coupling of the transformer is ideal, the relations

V3/V1=N3/N1

V3/V2=N3/N2

exist. V1 and V2 are voltages generated at M1 and M2, respectively. Also, V1 and V2 are provided from the power source 219. Then, the voltage V3 generated according to the timing at which the switches M1 and M2 turn to ON is applied to the probe 100.

In this example, the signals 216 and 217 having different frequencies are applied as input signals. Accordingly, M1 and M2 turn to ON at different frequencies, and the output signal is applied to the portion of the pulse transformer connected to the probe 100 at the timing that is a mixture of timings at which M1 and M2 turn to ON. When the input signals 216 and 217 are given, the output signal is as shown by a signal 218.

As has been described in detail above, the invention provides a square wave signal transmission circuit in which the amplitude of the output signal can be changed as desired by changing the duty ratio of the input signal. Furthermore, the square wave signal transmission circuit can output a signal having different frequency components in any combination ratio.

Although the preferred embodiments of the ultrasonic diagnostic apparatus and the like in accordance with the invention have been described with reference to the accompanying drawings, the invention is not limited to these embodiments. It is apparent to the person skilled in the art that various variations and modifications can be conceived without departing from the scope of the technical spirit disclosed herein, and also it is understood that those variations and modifications naturally fall within the technical scope of the invention.

### Description of Reference Numerals and Signs

00 ultrasonic vibrator, 01, 06, 09, 10 power source, switch circuit, 03 switch control unit, 04, 05, 14, 15, 16, 17 timing waveform, 100 probe, 101 element selector, 102 transmission/reception separator, 103 transmission processor, 104 transmitter, 105 reception amplifier, 106 phasing addition processor, 107 signal processor, 108 scan converter, 109 display monitor, 110 controller

1. An ultrasonic diagnostic apparatus, characterized by comprising:
an ultrasonic probe in which multiple ultrasonic vibrators for transmitting/receiving an ultrasonic wave are arranged;
a transmitter configured to provide an electric signal to each of the vibrators in the ultrasonic probe, the transmitter providing a square wave signal having any multiple frequency components to the each of the vibrators, causing the vibrators to form an ultrasonic beam;
a receiver configured to receive a reception signal obtained by transmitting the ultrasonic beam; and
a signal processor configured to form an ultrasonic image based on the reception signal.

2. The ultrasonic diagnostic apparatus according to claim 1, further comprising a switch section configured to variably set the duty ratio of the square wave signal provided to the each of the vibrators.

3. The ultrasonic diagnostic apparatus according to claim 2, wherein the switch section variably sets the duty ratio of the square wave signal with time.

4. The ultrasonic diagnostic apparatus according to claim 2, wherein the switch section sets the duty ratio of the square wave signal provided to the each of the vibrators differently for each vibrator.

5. The ultrasonic diagnostic apparatus according to claim 1, further comprising a controller configured to control the transmitter to output the square wave signal having multiple frequency components when tissue harmonic imaging is performed.

6. The ultrasonic diagnostic apparatus according to claim 2, further comprising a controller configured to control the square wave transmission circuit to variably control the duty ratio in the period at which the transmitter provides the square wave signal to the vibrator.

7. The ultrasonic diagnostic apparatus according to claim 2, wherein the controller further comprises a control unit for controlling the square wave transmission circuit for variably controlling from a first ON-duration set in the switch section to a second ON-duration that is different from the first ON-duration in the period at which the transmitter provides the square wave signal to the vibrator.

8. The ultrasonic diagnostic apparatus according to claim 2, wherein the controller further comprises a control unit for dividing the period at which the transmitter provides the square wave signal to the vibrator, providing the vibrator with multiple signals having different frequencies in those respective divided durations of the period, and controlling the square wave transmission circuit to variably control the duty ratio.

9. The ultrasonic diagnostic apparatus according to claim 4,
wherein the transmitter is connected to positive and negative power sources, and
wherein the positive and negative power sources include multiple power sources.

10. The ultrasonic diagnostic apparatus according to claim 9, characterized by further comprising a control unit configured to control the multiple positive and negative power sources by the switch section.

11. The ultrasonic diagnostic apparatus according to claim 1, wherein the transmitter includes a single power source and a pulse transformer.
